(19) United States
(12) Patent Application Publication
(10) Pub. No.: US 2023/0255846 A1
(43) Pub. Date: Aug. 17, 2023

(54) LIMB POSITIONING DISPOSABLES WITH INFLATABLE BLADDERS

(71) Applicants: Smith & Nephew, Inc., Memphis, TN (US); Smith & Nephew Orthopaedics AG, ZUG (CH); Smith & Nephew Asia Pacific Pte Limited, Singapore (SG)

(72) Inventors: Philip Cormier, Newburyport, MA (US); Cassandra Rapp, Woburn, MA (US); Luis Davila, Chelmsford, MA (US); William D. Obendorf, Chelmsford, MA (US); Michael Bridgers, Andover, MA (US)

(21) Appl. No.: 18/015,867

(22) PCT Filed: Jul. 14, 2021
(86) PCT No.: PCT/US2021/041662

§ 371 Date: Jan. 12, 2023

Related U.S. Application Data
(60) Provisional application No. 63/051,469, filed on Jul. 14, 2020.

(51) Int. Cl. A61G 13/00; A61G 13/12

(52) U.S. Cl. CPC A61G 13/0045; A61G 13/1265; A61G 13/1235; A61G 13/124

(57) ABSTRACT

An orthopedic limb interface device is disclosed for positioning and retaining a lower portion of the arm of the human body during orthopedic operatory procedures. The device includes a handplate coupled to a sleeve. The sleeve surrounds and grips the arm and is lined with an inflatable bladder. The bladder includes a plurality of inflatable zones, at least one zone having inflation limiting means such that the inflated bladder has a variable inflation profile therealong. The variable inflation profile is configured to increase retention at target areas of the limb, while softly retaining other areas of the limb to avoid limb numbness. Areas of increased retention may engage portions of the arm to improve the grip on the arm for reliable limb distraction.

### CROSS-REFERENCE TO RELATED APPLICATIONS

This application claims benefit to and incorporates by reference in its entirety, U.S. Provisional Patent No. 63/051,469; filed Jul. 14, 2020 and titled “LIMB POSITIONING DISPOSABLES WITH AIR BLADDERS”.

### FIELD OF THE INVENTION

This disclosure relates generally to the field of limb interface devices that retain and position a limb during orthopedic arthroscopic and arthroplasty surgery via limb positioning devices.

### BACKGROUND

During orthopedic arthroscopic and arthroplasty surgery, it is often necessary to position a patient's limb to obtain access to the pathology. Traction may also be necessary to distract the joint to obtain access to certain anatomy. To position the limb, a Limb Interface Device (LID) engages the patient limb and connects to a surgical positioning arm, which acts as a mobile anchor point. The LID preferably arrives at the operating room sterile.

Current LIDs may be specific to the limb and position of the patient and the surgery being performed. For example, a prior art LID may include a surgical sleeve or wrap 10 is illustrated in FIG. 1A. Sleeve 10 attaches a patient's arm 2 and hand 4 to a limb-positioning arm 20. In this example, before being secured to the limb positioning arm 20, the patient's hand 4 is attached to a handle or bar 6 and secured into a balled fist, as shown in FIG. 1A. Portions of the patient arm 2 and hand 4 are draped or covered in a flexible sleeve 10 and secured in place with straps 5. Sleeve 10 may include open-cell foam material for padding. A surgical sleeve or wrap 10 may also be used with the limb positioning arm 20 in a different orientation to position the patient's arm, such as the beach chair position shown in FIG. 1B.

Current LIDs may only be available in one size, so the fit between the patient and LID varies. This variation in fit can lead to movement and slip between the patient and LID. This slip is problematic as it can result in a loss of distraction required to perform the procedure thereby increasing surgery time while the distraction space is reestablished. Having a device too tight to compensate for slip however, can lead to numbness in the patient's hand 4 and fingers 1.

Current LIDs must be provided sterile and the patient limb must be sterilized with a sterile solution from the operative site to the distal end of the limb. For a shoulder procedure, the sterile solution would be applied from the shoulder and down the arm to the fingertips. The sterile solution then requires time to dry before the limb interface device can be attached to the patient. If a sterile solution was not applied all the way down to the fingertips, a stockinette is placed on the patient first to ensure sterility is maintained while using the limb interface device. The patient limb is then placed into the LID, the LID may close via a securing mechanism like Velcro, and then a self-adhering wrap like Coban may be wrapped around the LID to secure the limb further. The operative limb is then covered with a sterile drape (not shown) so that only the surgical site is exposed. This procedure takes time and requires multiple steps and components.

Therefore, there is a need for a LID that accommodates more limb sizes, and can adjustably fit and grip in key locations along the limb for better retention and distraction without causing limb numbness. There is also a need for an LID that requires less time and reduced individual components to prepare a positioned and sterilized patient limb.

### BRIEF DESCRIPTION OF THE DRAWINGS

For a detailed description of example embodiments, reference will now be made to the accompanying drawings in which:

FIG. 1A illustrates a patient arm being supported by an LID that is known in the art, the patient arm positioned in a “Lateral Decubitus” position;

FIG. 1B illustrates a patient arm being supported by an LID that is known in the art, the patient arm positioned in a beach chair position;

FIG. 2A illustrates the anatomy of an anterior side of an arm, wrist and hand;

FIG. 2B illustrates the anatomy of a medial side of an arm, wrist and hand;

FIG. 3A illustrates an LID embodiment with an inflatable bladder in a wrapped configuration, in accordance with the present disclosure;

FIG. 3B illustrates a side view of the LID embodiment illustrated in FIG. 3A, in accordance with the present disclosure;

FIG. 3C illustrates a view of an underside of the LID embodiment illustrated in FIG. 3A, in accordance with the present disclosure;

FIG. 3D illustrates an inflatable bladder of the LID embodiment illustrated in FIG. 3A, the bladder in an unwrapped configuration with zones of quilting dots, in accordance with the present disclosure;

FIG. 3E represents varying zones of inflation of the inflatable bladder illustrated in FIG. 3D, in accordance with the present disclosure;

FIG. 3F illustrates a cross section of zones of the inflated bladder illustrated in FIG. 3D with inflation limiting means in accordance with the present disclosure;

FIG. 3G illustrates a cross section of zones of the inflated bladder illustrated in FIG. 3D, the zones absent inflation limiting means in accordance with the present disclosure;

FIG. 3H illustrates lateral and medial portions of an inflatable bladder illustrated in FIG. 3D on corresponding sides of a patient arm, hand and wrist, in accordance with the present disclosure;

FIG. 3I illustrates the varying inflation profile of posterior and anterior portions of the inflatable bladder illustrated in FIG. 3D on corresponding sides of a patient arm, hand and wrist, in accordance with the present disclosure;

FIG. 3J illustrates a view of the LID embodiment illustrated in FIG. 3A in an unwrapped configuration, in accordance with the present disclosure;

FIG. 3K illustrates a view of an underside of the LID embodiment illustrated in FIG. 3A in an unwrapped configuration, in accordance with the present disclosure;

FIG. 4A illustrates another LID embodiment with an inflatable bladder, in accordance with the present disclosure;

FIG. 4B illustrates the LID embodiment illustrated in FIG. 4A wrapped around a patient arm and wrist, with a sterile drape preassembled to the LID and covering the patient arm and LID, in accordance with the present disclosure;

FIG. 4C illustrates an example bellows and rib pattern of the inflatable bladder illustrated in FIG. 4A, the inflatable bladder shown in an open configuration, in accordance with the present disclosure;

FIG. 4D illustrates a side view of the LID embodiment shown in FIG. 4A, in accordance with the present disclosure;

FIG. 4E illustrates a top view of the LID embodiment shown in FIG. 4A, in accordance with the present disclosure;

FIG. 5A illustrates another LID embodiment with an inflatable bladder, in accordance with the present disclosure;

FIG. 5B illustrates a side view of the LID shown in FIG. 5A, in accordance with the present disclosure;

FIG. 5C illustrates an example inflation limiting zone of the inflatable bladder in the LID illustrated in FIG. 5A, the bladder shown flattened and removed from the example LID shown in FIG. 5A, in accordance with the present disclosure;

FIG. 6A illustrates another LID embodiment, in accordance with the present disclosure; and

FIG. 6B illustrates a side view of LID shown in FIG. 5A, in accordance with the present disclosure.

### SUMMARY

Disclosed herein is an LID that includes one or more inflatable bladders to hold a patient limb. The bladder inflates to varying degrees to improve functionality. For example, the bladder may inflate the most around target areas on the limb to improve limb retention given the external loads on the limb. This may improve the grip between the patient and the LID during positioning and traction on the limb. Concomitantly, the bladder may include means to constrain inflation along other areas, and thus relieving these areas on the LID, thus mitigating the likelihood of numbness or nerve damage to the patient limb. For example, the inflatable bladder may preferably inflate more on lateral and medial sides of the wrist while minimally inflating adjacent the anterior surface of the wrist, which may grip around the wrist while providing pressure relief along the nerves extending along the wrist anterior side. The inflatable bladder may be inflated via a pump, integral with the bladder.

An incorporated inflatable bladder with means to inflate more in some zones and less in others may provide a customizable fit to accommodate a range of patient anatomy and sizes, as the fluid fills in any of the gaps between the patient and limb interface device, therefore limiting the amount of slip between the two. An integrated sterile barrier drape may allow an unsterilized hand to be placed into the device without the use of a stockinette or Coban, which saves pre-op time.

Used herein is the term “Sleeve” defined as a construct that carries the inflatable bladder and constrains inflation of the inflatable bladder radially away from limb. Sleeve is configured to assemble over and along the limb and hold the bladder against the limb. Sleeve may be a tube, wrap, sheath, cuff or clamshell. Sleeve may be provided as a tube that is assembled by sliding over a free end of the limb to encircle to limb. Sleeve may be a wrap that is provided in an open unwrapped form and assembled by wrapping the sleeve around the limb. In this example, sleeve may include closing means such as Velcro or adhesive tape or hook and eyes. Sleeve may be a clamshell and may include at least two mating segments that move relative to each other to provide an opening to thread the limb therein. Sleeve may include a hinge or snap. Sleeve may be formed of a rigid and/or flexible material. Sleeve may be formed of a knitted textile.

Disclosed herein is an orthopedic limb interface device that may position and retain a lower portion of the arm and hand of a human body during orthopedic procedures. It may include a handplate that may be rigid and may couple directly to a positioning arm. It may also include a sleeve coupled to the handplate. Sleeve may be assembled to the lower portion of the arm and in assembled form, encircle the arm and the wrist. The sleeve may include an inflatable bladder, which may be inflated to adjust an internal opening size of the sleeve and thereby adjust to the arm and wrist contours. The inflatable bladder includes a plurality of inflatable zones that may be fluidly coupled to each other, forming zones of increased grip on the arm and wrist and zones of pressure relief on the arm and wrist. At least one of the zones defines a zone of greater inflation for increased grip on the arm and wrist. At least one of the zones defines a zone of limited inflation along the bladder for pressure relief to avoid numbness to the arm and hand. The plurality of zones are configured to improve retention of the orthopedic limb interface device with the arm.

In some example embodiments, one of the zones of greater inflation may be disposed along an end of the bladder, configured to engage and encircle a wrist to improve the grip on the arm and more specifically provide increased and targeted resistance to slip upon axial traction along a longitudinal axis of the arm. In some example embodiments, the plurality of inflatable zones may include at least two discrete zones of greater inflation separated by at least one zone of limited inflation. The at least two discrete zones may be configured to engage opposing sides of an anterior surface of the arm, such that when surrounding the arm the two discrete zones are configured to improve a resistance to slip upon application of an external rotational force to the arm. At least one zone of limited inflation may be defined by means of limiting inflation including at least one of ribs, quilting or bellows. At least one zone of limited inflation may be at least partially defined by a plurality of quilting dots. The device may also include a sterile drape coupled thereto, configured to unfold and provide a sterile barrier over the arm, handplate and sleeve. The bladder may be provided integral with and operatively coupled to a fluid transport system, configured to transfer fluid into the bladder and thereby inflate the bladder, the fluid transport system including a pressure-limiting valve.

Another orthopedic LID embodiment is disclosed that positions and retain a lower portion of the arm and hand of the human body during orthopedic procedures. This device includes a handplate coupled to a sleeve. The sleeve is configured to surround and grip the arm and may be lined by a bladder configured to inflate upon the introduction of a fluid therein and adjust an internal opening size of the sleeve. The bladder includes a plurality of inflatable zones that may be fluidly coupled to each other, including at least one zone with inflation limiting means to limit inflation and at least one zone free of inflation limiting means configured to inflate more so than the at least one zone with inflation limiting means. The plurality of inflatable zones are configured to improve retention of the orthopedic limb interface device with the arm.

In some example embodiments, at least one of the zones including inflation limiting means reduces pinching of the arm and thereby mitigates numbness of the hand. At least one zone free of inflation limiting means may extend along a distal end of the bladder, and may inflate and engage a wrist portion of the arm to improve a resistance to slip upon axial traction along a longitudinal axis of the arm. The at least one zone free of inflation limiting means may include two discrete elongate zones that may extend axially along the bladder, separated by a zone including limited inflation means. The two discrete zones may engage the patients arm and improve a resistance to slip upon application of a rotational force to the arm. The inflation limiting means may include includes at least one of ribs, quilting or bellows. The inflation limiting means may include a plurality of quilting dots. The orthopedic limb interface device may also include a sterile drape coupled thereto, configured provide a sterile barrier over the arm, handplate and sleeve. The bladder may be operatively coupled to a fluid transport system, configured to transfer fluid into the bladder and thereby inflate the bladder, the fluid transport system including a pressure-limiting valve.

Another orthopedic limb interface device is disclosed herein that positions and retains the lower portion of the arm and wrist of the human body during orthopedic procedures. The device includes an inflatable bladder operatively coupled to a handplate, the inflatable bladder configured to encircle the arm and wrist and inflate upon the introduction of a fluid therein. The inflatable bladder includes zones with inflation limiting means and zones absent inflation limiting means so that the inflatable bladder defines a variable inflation profile. The variable inflation profile provides increased retention of targeted portions of the wrist and hand to rests slipping of the arm while under traction. The variable inflation profile may include at least one zone absent inflation limiting means disposed along a distal end of the inflatable bladder configured to encircle a wrist of the patient arm and retain the orthopedic limb interface device while undergoing axial traction. The variable inflation profile may include at least two zones absent inflation limiting means, disposed along a longitudinal axis of the inflatable bladder. These two zones may engage along a length of the patient arm and retain the orthopedic limb interface device while undergoing an external rotational traction. The inflation-limiting mean may include a plurality of quilting dots. The variable inflation profile may include a first zone including a plurality of dots having a first spacing relative to each other, and a second zone including a plurality of dots having a second, different spacing relative to each other than the first zone, and thereby forming a variable inflation profile between the first and second zones.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open-ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

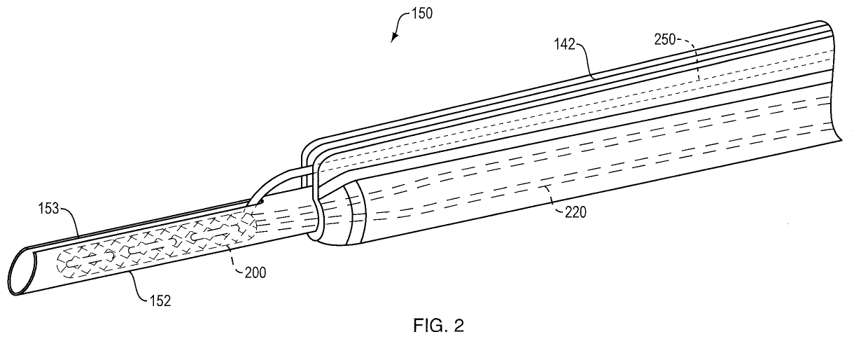

The hand and forearm anatomy is illustrated in FIGS. 2A and 2B, including the fingers 1, arm 2, wrist 3 and hand 4. The carpal area 7 is at the proximal end of the hand 4 and includes the scaphoid and pisiform bones therein. The proximal end of the first metatarsal 8 is located adjacent the distal end of the carpal area 7. Disclosed herein are LIDs in the forms of sleeves, clamps or wraps that enclose and couple to a positioning arm 20 and engage the various portions of a patient's arm 2, wrist 3 and hand 4 to help position the limb. The LID includes at least one bladder configured to inflate and conform to the contours of the arm 2, wrist 3 and hand 4 for an improved grip on the limb while avoiding or reducing overly pinching and causing numbness or limb damage. The bladder may be tubular or conical in shape, once arranged around the limb. The bladder is configured to expand preferentially into portions of the arm 2, wrist 3 and hand 4 for an improved retention on the limb during traction. The bladder may be inflated with fluid, to conform to the limb shape. The bladder may preferentially expand to a greater depth or larger profile in some zones, while remain smaller in profile in other areas. Stated another way, the bladder may include at least one low profile zone and at least one large profile zone. While each zone may be fluidly separated, and thereby formed from discrete individual bladders that are inflated one at a time, it is preferable that they are fluidly continuous with each other. Each zone may have a consistent inflation depth when fully expanded, or may have a varying inflation depth therealong. It should also be understood that bladders may be formed by sealing at least two sheets of material around a peripheral border, and adjacent these sealed borders the inflation profile may gradually taper and therefore vary, this taper is different from the intention of the zone.

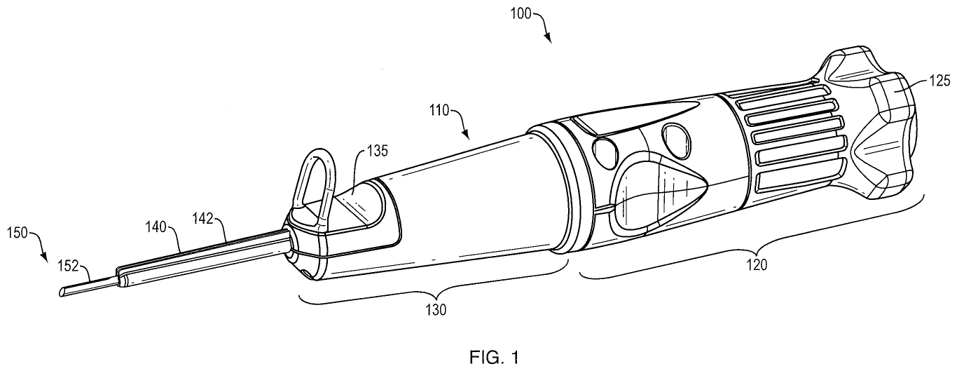

In this example, LID retention may be improved during axial traction along the limb longitudinal axis, typical in the lateral decubitus position. The bladder may include quilting or bellows in the lower profile zones to limit the inflated profile and reduce the volume of fluid required to inflate these zones. The bladder may expand to a larger profile at two discrete portions that are circumferentially spaced from each other. This may improve the device retention during rotational forces on the limb, and inhibit the LID from rotating around the patient limb. This rotational force is more common when in the beach chair position shown in FIG. 1B.

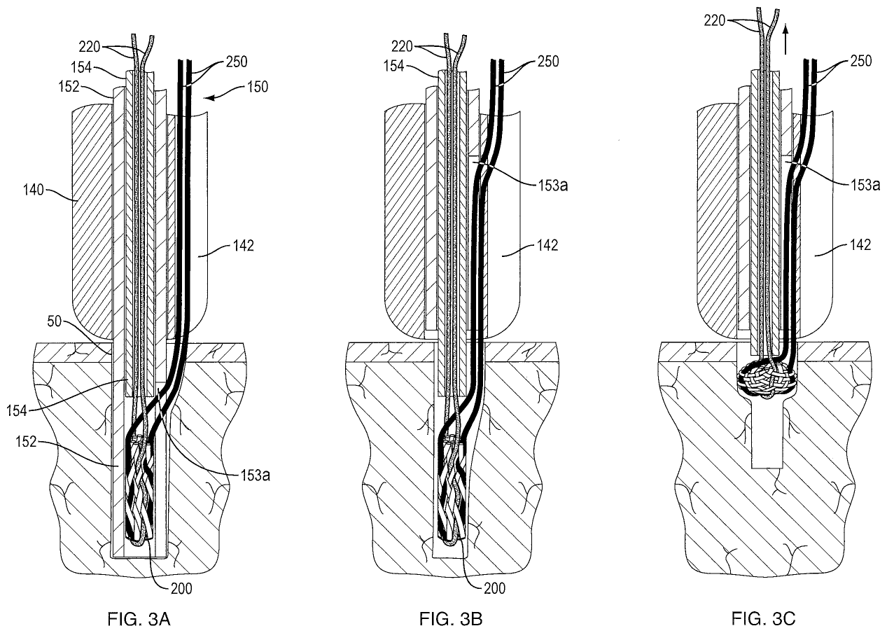

A first example device is illustrated in FIG. 3A-3K. LID 200 may include a handplate 220 and sleeve in the form of a wrap 230 attached thereto. FIG. 3A-3C illustrate the LID 200 with the wrap in a closed (wrapped configuration). Handplate 220 is generally configured to provide some rigidity to the LID 200 and may support the arm 2, hand 4 and wrist 3. Handplate 220 may engage an anterior side of the patient's arm. Handplate 220 may be formed of a rigid material such as a plastic or metal. Handplate 220 may include a means to couple to a patient positioning arm 20 (best seen in FIG. 1B). For example, the handplate 220 may include a post 210 for coupling to positioning arm 20. The patient's hand 4 and fingers 1 may lie on contoured surface 222 of handplate 220 with the carpal area 7, wrist 3 and arm 2 enclosed by wrap 230. Contoured surface 222 may be lined with a layer of viscoelastic foam 225 that has a high coefficient of friction to reduce the potential for slip between the patient arm 2 and the LID 200.

Wrap 230 is configured to wrap around and grip a patient's arm 2, wrist 3 and carpal area 7. More specifically wrap 230 may be assembled to encircle the patient's hand 4, wrist 3, and arm 2. Wrap 230 may include an outer layer 236 formed of a flexible material such as a knitted textile. External layer 236 may define the entire outer surface of the wrap 230, as assembled, and thus direct inflation of the inflatable bladder 240 inwards towards the internal opening of the wrap 230, as assembled. Outer layer 236 may include securing or closing means 247 attached thereto, such as Velcro or hooks and eyes.

Wrap 230 may have a tubular portion 230a and distal portion 230b. Tubular portion 230a is generally configured to engage the arm 2 tightly, while the distal portion 230b may lightly engage the hand 4 to keep it on the handplate surface 222. Distal portion 230b may enclose around the first metacarpal area of the patient's hand 4. Distal portion 230b may preferably loosely grip the hand 4. Tubular portion 230a defines a first end 232 configured to circumferentially extend around the smaller cross section area of the wrist 3. Tubular portion 230a defines a second, opposing end 233 configured to circumferentially extend around the larger cross section area of the patient forearm. More specifically, end 232 preferably engages the area adjacent the proximal end of the first metacarpal 8 and second end 233 is approximately 3 inches proximal from the wrist joint 3 along the forearm. Tubular portion 230a may be conical shape, such that end 233 may define a longer edge or larger opening than end 232 to accommodate the larger cross section of the forearm relative to the wrist 3 and carpal area 7. Ends 232 and 233 may each define edges that are parallel to each other and may be curved (best seen in FIG. 3D). When wrapped around the arm 2, the wrap 230 may tightly engage the arm and conforms to the arm shape.

FIGS. 3D-3I illustrate various aspects of the wrap with the handplate removed to simplify the figures. Starting with FIG. 3D illustrating the wrap 230 and inflatable bladder 240 in a flattened, unwrapped configuration includes at least one. Wrap layer 236 may be lined with at least one inflatable bladder 240, such that upon wrapping around the arm 2, the bladder 240 engages the patient skin. Outer layer 236 may contain the radial inflation of the bladder 240 to direct inflation preferably inwards to reduce the size of the internal size of the wrap 230, in the wrapped configuration. Bladder 240 is configured to selectively receive a fluid therein and radially expand. The radial expansion may be preferentially directed inward towards the patient limb, directed at least partially due to restriction from the external layer 236. In some embodiments, an outer layer of the bladder may be formed of a more rigid material. Selectively inflating the bladder 240 is configured to improve the grip on the patient limb. Bladder 240 may be fluidly coupled to a pump 245 that may be hand operated to inflate the bladder 240.

Shown in at least FIG. 3D and FIG. 3F, a pump 245 may be integral with bladder or wrap 230. Example locations of pump 245 are shown in both FIGS. 3A and 3D, that are shown in different locations. Pump 245 may be covered by a sterile drape (discussed later) and needs to be easily discoverable and accessible. In FIG. 3A pump is on a posterior side of arm 2 as assembled, which may be preferable while positioning the patient similar to a beach chair position, as illustrated in FIG. 1A. In FIG. 3D, pump is on an anterior side of arm 2, along the patient forearm, which may be preferable for patient positioning similar to the lateral decubitus position, illustrated in FIG. 1A. Pump 245 may be manually operated pump and may include a check valve. The pump may include a fluid chamber having a domed surface 246. A fluid flow conduit (not shown) may be fluidly coupled to fluid chamber, the conduit including a valve having an inlet and outlet. Domed surface 246 may be deformable by hand to pump fluid through the fluid conduit and into the bladder 240. The check valve may have at least one exhaust channel to provide one-way fluid flow from the fluid chamber through the exhaust channel. In other embodiments, the bladder may operatively couple to a fluid cartridge, such as a CO2 cartridge and may inflate upon actuation of the cartridge. In some embodiments, the pump 245 may be configured to limit over-pressurization of the air bladder 240. For example, the pump may be configured such that the efficiency of the pump 245 decreases with each pump application and eventually each successive pump will no longer practicably increase the pressure in the bladder. The bladder 240 is considered in an over-inflated condition when its internal pressure exceeds 5 psi.

In some embodiments, portions of the bladder 240 may include means to reduce the volume of fluid required to inflate the bladder 240 as a whole. These means may include ribs, bellows or quilting. In some embodiments, portions of the bladder 240 may include means to preferably constrain some zones of the bladder 240 more than other zones. For example, the bladder 240 may define a single enclosed volume fluidly coupled to the pump 245, the volume locally reduced in some zones by at least one quilting dot 242. A quilting dot 242 may be formed thermally or adhesively, joining opposing inner surfaces of the bladder walls at a discrete location, defining a dot 242. Where the dots 242 are absent or spaced further apart (lower density), the bladder 240 may inflate further, and thereby reduce an internal size of the corresponding zone of the wrap 230 more so than were the quilting dots 242 are spaced closer together. As shown, all the quilting dots 242 may be the same size. Areas targeted for less inflation may also include larger sized quilting dots.

In the example shown in FIG. 3D, the quilting dots 242 are absent or lower in density along end 232. End 232 is configured wrap around the area adjacent the pisiform and tubercle of the scaphoid. When wrapped around the patient arm and wrist, this end 232 may be wrapped tightly around the carpal area 7, and then preferentially inflated to improve the grip around the carpal area 7 and wrist 3. This combination is configured to inhibit the hand from slipping out when an axial force is applied, for example during distraction. When the bladder 240 inflates in this area, it matches the circumferential contour of the carpal bones, and therefore prohibits the hand from slipping through the disposable. This is especially valuable for resisting axial forces along the limb, typical in lateral decubitus shoulder and elbow positions.

In some embodiments, end 232 may include portions that preferably inflate more than other portions. While the overarching preference is to inflate to a smaller resultant internal opening, avoiding pinching of the anterior and posterior sides of the wrist may be preferably, to reduce hand numbness. Therefore, optional quilting may be added (242a) adjacent end 232 corresponding with anterior and posterior placement around the wrist 3.

Distal portion 230b may be inert, in that it may not be inflatable. In other embodiments, bladder 240 may extend into distal portion 230b, and may include inflation restricting means, such as quilting 242, to reduce the amount of fluid required to inflate bladder 240 as a whole. Distal portion 230b is configured to loosely wrap and retain thumb and hand 4 on handplate 220, and a tight grip is not preferable. In other embodiments, distal portion 230b may be separately formed from bladder 240. It may be removable, or directly coupled to handplate 220. Wrap 230 may include an inert flap 247 that is inert such that it does not inflate. Flap 247 may overlap a portion of the wrap 230 and secure the wrap 230 around the limb. Flap 247 may include securing means such as Velcro or adhesives to secure the wrap around the patient limb.

A simplified example representation of different zones are illustrated in FIG. 3E, illustrating different zones of bladder 240. Zone A may extend along end 232 configured to inflate to a reduced resultant internal size of the wrap as assembled, and thereby engage an external surface of the wrist 3. Zone A may extend circumferentially around the wrist 3 and may include a variable inflation profile therealong, providing more inflation along lateral and medial surfaces of the wrist 3, with some inflation limiting means disposed at zones A′. Zones A′ may provide pressure relief along anterior and poster portions of the wrist 3 and reduce hand numbness. Zones B may have a low density of quilting dots 242 or be free of any inflation limiting means for preferential inflation. Zones B for example may define a plurality of discrete zones, circumferentially spaced from each other when wrapped around the patient limb. Zones B may define at least two elongate zones that extend between the two ends 232 and 233 and may extend along the anterior sides of the forearm 2 between the wrist 3 and end 233. In other embodiments, zones B may engage the patient forearm, lateral to the handle plate 220 and may engage medial and lateral sides of arm 2. The absence of quilting or bellows in this area allows the bladder 240 to preferentially inflate further, such that it holds the forearm securely against the handplate 220, to prevent the LID from rotating around arm 2. This support is especially critical for beach chair shoulder and elbow procedures. Zones C may define areas of high-density inflation limiting means. Zones C may include a plurality of quilting dots 242 that are configured to reduce inflation therelong. Quilting dots 242 may vary in size and frequency or spacing to provide a variable inflation profile upon inflation of the bladder 240. Distal portion 230b may also define zone D, which may not inflate at all, or may include a plurality of inflation limiting means to minimally engage the patient's hand.

Example cross sections of wrap 230 in an inflated configuration are illustrated in FIGS. 3F and 3G, with reference to FIGS. 3D and 3E for locations and zones. External layer 236 may line the bladder 240. Zone A may preferentially inflate to a larger size than zone C, forming a smaller internal opening when wrapped around the patient arm 2. Zone C may be limited by a plurality of quilting dots 242. Zone B may define an elongate zone that engages lateral sides of the arm 2. Zone D may be inert.

FIG. 3H illustrates an anterior view of the arm 2 with representative portions of the wrap 230 shown. For example with wrap 230 engaged around the arm, zones C may extend along and engage the lateral and medial surfaces of the arm 2. Two discrete zones B may extend along the anterior side. Zones A with less inflation limiting means may engage contours of writs 3. FIG. 3I illustrates a side view of the arm 2 with zones C and zones A′ represented along the arm 2 up to the carpal area 7. Zones A′ may include inflation limiting means to relief pressure on the nerves that extend along the anterior side of wrist 3.

In some embodiments, wrap 230 may include a plurality of bladders that may be independently inflated. For example, the bladder may include two bladders split just proximal to where the wrap 230 may engage the carpal bones. Alternatively, the two bladders may be fluidly connected via a bridging channel so that only one pump and inlet valve is needed to inflate both portions of air bladder assembly.

FIGS. 3J-3K illustrate the LID 200 with the wrap 230 in a flat configuration. Handplate 220 may include two connecting post, such as posts 210a, 210b. Post 210a may couple to positioning arm 20 for a patient lateral decubitus position for example while post 210b may be a preferable location for coupling to a positioning arm 20 for a patient in a beach chair position. Handplate 220 may include a proximal leg 221 that defines a contoured surface that supports the patient arm 2. Wrap 230 may be operatively coupled to proximal leg 221. Wrap 230 may be sandwiched between two layers 221a and 221b of leg 221. During assembly, the two layers 221a, 221b may be separated and a portion of wrap 230 placed between them before fixedly coupling the two layers 221a, 221b. Coupling may be via an adhesive, ultrasonic welding, heat sealing or snap fits for example. This sandwiched portion of the bladder 240 and bladder area immediately surrounding the sandwiched portion of the wrap 230 may include inflation limiting means, such that inflation of the bladder 240 adds minimal stress on the layer coupling. Sandwiching the wrap 230 may preferably seal the bladder 240 to prevent expansion in that area. An example coupling portion 231 of the wrap 230 is illustrated on FIG. 3D. In some embodiments, the handplate 220 may include a plurality of channels 223 formed therein, in between the two layers 221a and 221b. Channels 223 may define fluid conduits to pass to both sides of the bladder 240 reducing stresses on the handplate 220. In some other example embodiments, a pair or series of bosses on the handplate located at the proximal and distal ends of the bladder may be used to keep the bladder from being pinched closed, allowing fluid to flow from one side of the bladder to the other. In some embodiments, the bladder 240 may be secured directly to the handplate 220 via a joining process, such as ultrasonic welding or the addition of an adhesive. In some embodiments, the handplate 220 may be lined with a viscoelastic foam 225 that has a high coefficient of friction to reduce the potential for slip between the patient and the LID. In some embodiments, the skin-contacting portion of the wrap 230 may be lined with a high coefficient of friction foam or fabric to reduce slip potential. In some embodiments, the skin contact portion of the bladder 240 has a surface finish with a high coefficient of friction to reduce slip between the patient and the limb interface device. The bladder material may be manufactured with a variety of different surface finishes, e.g., matte or clear, to produce differing coefficients of friction. Bladder 240 may be formed of vinyl. Bladder 240 may be inflated with a gas or liquid. Bladder 240 may be preferably inflated with air.

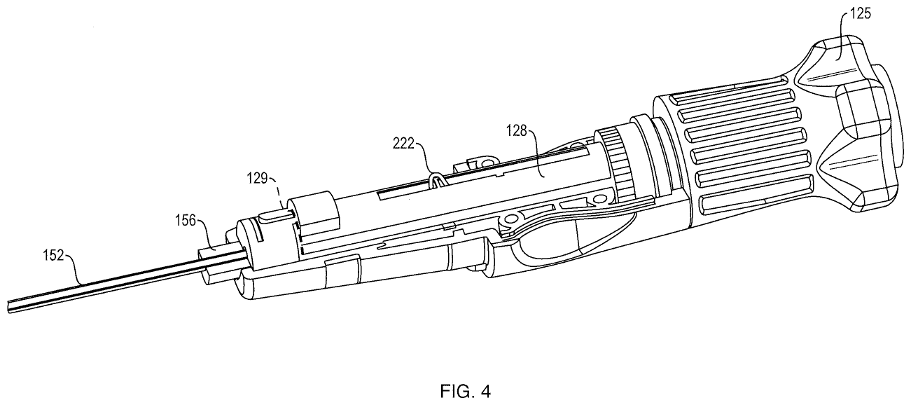

Another example embodiment of an LID 300 with an inflatable bladder 340 is illustrated in FIG. 4A-4E. LID 300 may be similar to LID 200 and like components are given the same numerical designation. LID 300 may include a handplate 220 operatively coupled to sleeve in the form of a tube 330 lined with an inflatable bladder 340. In this embodiment, other example inflation limiting means are disclosed including bellows 342 and ribs 343. Tube 330 may be provided as a continuous conical tube and therefore slides over the hand 4 and onto patient's wrist 3 and arm 2. Tube 330 is preferably positioned with end 332 adjacent the proximal end of the first metacarpal of the hand 8 to approximately 3 inches proximal to the wrist joint along the forearm, as shown in FIG. 4B. In this embodiment, the tube 330 may be provided as a complete enclosed tube 330, thus avoiding the need to wrap the bladder 340 around the patient arm and therefore avoiding securing means such as Velcro or hooks and eyes. In this embodiment, bladder 340 may include bellows 342 to limit the inflation profile of the bladder 340. Bellows 342 may be similar to quilting dots, in that they are configured to locally interrupt the bladder volume at discrete locations to reduce the required volume of fluid to inflate the bladder overall. Bellows 342 also direct areas for greater inflation and areas for reduced inflation to improve the grip on the limb. Bellows 342 may define linear or elongate interruptions to the bladder internal volume that extend along a length of the bladder 340, compared with quilting dots that define individual smaller or shorter interruptions. Bellows 342 may be formed be sealing lines of the bladder lining together along orientations that direct inflation for the targeted inflation zones. Bellows 342, similar to quilting dots 242, preferably terminate at locations spaced away from the outermost boundary of the bladder, and thus preferably allows fluid to flow around the ends of the bellows 242 to fill the entire bladder 340.

FIG. 4C illustrates a tube 330 transected and flattened to illustrate the inflation limiting means and their example locations. The handplate 220 has been removed for ease of understanding the figure. In this embodiment, tube 330 may form a cone, with a narrower end 333 adjacent the forearm 2 of the patient and a larger opening adjacent the patient's thumb. Similar to other embodiments, bladder 340 may include a pump 245 integral with the bladder 340 that may be disposed at the narrower end 333. Other locations as discussed herein are also contemplated. Pump 245 is located to be readily accessible through a sterile drape. A plurality of seal lines forming the bellows 342 are illustrated where the internal wall of the bladder has been sealed along said lines 342. Bellows 342 are configured to limit the inflation volume required to inflate the bladder 340. Bellows 342 are preferable absent or less frequent along the zone adjacent end 332 that may correspond to a location around the wrist 3, defining a zone similar to zone A as previously disclosed herein. Bellows 342 may define lines that extend along and across the bladder 340. Bellows 342 may be formed from lines oriented along the patient's arm and along a longitudinal axis of the tube 330. Bellows 342 are formed by adhering lines of opposing sides of the bladder together. Specifically, the bellows 342 may preferably be located on the posterior portion of the arm. This limits the amount of fluid required to inflate the bladder 340 as a whole while preferentially inflating the zones of the bladder 340 located on the medial and lateral portion of the wrist 3. As explained herein, this limits the slip between the patient and limb interface device.

In addition, bladder 340 may include pockets 345 for receiving ribs 345 therein. Pockets 343 may be disposed on an external surface of the bladder 340, and may be placed on portions of the bladder 340 that lie over the posterior and anterior portion of the wrist 3 and arm 2. Illustrated in FIG. 4C, an external surface of tube may include pockets 343 that slideably receive ribs 345 therealong. These ribs 345 direct inflation into the arm 2 and limit the amount of fluid required to inflate the bladder 340 as a whole. Ribs 345 may be formed of ripstop nylon or Dacron. These ribs 345 allow for flexing or inflation of the bladder 340 perpendicular to the longitudinal axis of the limb, but limit or even prevent flexing of the bladder 340 parallel to the longitudinal axis of the limb. Ribs 345 inhibit the bladder 340 from balling in on itself to ensure that the bladder contacts and retains the critical anatomy of the patient so that the device remains secured to the patient while under traction. In some embodiments, there may be a release valve to deflate the bladder in order to remove it from the patient.

In some embodiments, a sterile barrier drape 370 may be operatively coupled to portions of the LID 200 or 300, example shown in FIG. 4B. For example, a sterile barrier drape 370 may be sealed along the distal portions of the handplate 220 such that it creates a sealed sterile barrier and the patient's hand 4 and fingers 1 may not require sterilization prior to loading the patient into the limb interface device. This may reduce the preparation time of the limb. The drape 370 may be translucent so that the color and condition of the patient's fingers 1 may be monitored as part of monitoring the patient's circulation throughout the duration of the procedure. The patient's pulse at their fingers may also be monitored by an either integral or removable pulse/heartrate sensor. The drape 370 may be sealed along the proximal portions of the handplate 320 such that it can be pulled up on the patient's forearm, proximal to the elbow, and onto the patient's upper arm to provide a sterile barrier along the patient's arm. When the drape 370 is folded in its as delivered configuration, the exposed face of the drape is the same face that will ultimately be in contact with the patient's skin upon drape deployment. This ensures any unintentional contact with the drape by an unsterile object (like the patient's hand during insertion) will not create a scenario where sterility of the final outwardly facing surfaces is compromised. The proximal end of the drape may include a strip of tape or other non-permanent adhesive strip (e.g. Coban) to secure the drape to the patient once deployed.

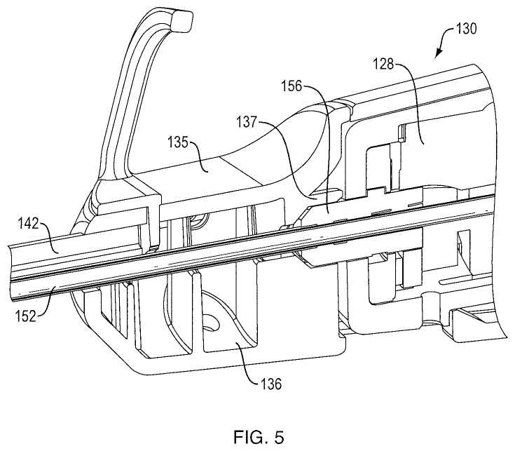

FIG. 5A-5C illustrates another embodiment of an LID 400 including a handplate 420 with a clamshell 430, lined with an inflatable bladder 440. Inflatable bladder 440 may only line the top portion of the clamshell 430. Bladder 440 may engage posterior, lateral and medial sides of the arm 2 and wrist 3. The clamshell 430 may be rigid and may be formed of plastic. Clamshell 430 may include a top 430a and bottom portion 430b, such that the bottom portion 430b extends along the curvature of the handplate 420. Bottom portion 430 may be continuous and integrally formed with handplate 420. Top portion 430a may follow the curvature of the posterior side of a patient's hand, wrist, and forearm. Clamshell 430 may include hinges 432 that allow the clamshell to hinge from the top or hinge from the side, to slide arm therein. Bladder 440 may line the inner surface of the top portion 430a such that the clamshell wall limits the bladder 440 from inflating radially away from the patient's hand and forces the expansion down around the patient's hand/wrist. In other embodiments, bladder 440 may line the inner surface of both the top and bottom portions of the clamshell. The pump 245 may be integrated into the top portion 430a of the plastic clamshell. Bladder 440 may include partial or entire areas of bellows and quilting as described herein, to reduce the amount of fluid required for inflation of the bladder 440. Specifically, the bellows and/or quilting may be disposed adjacent the posterior portion of the hand. This directs the portions of the bladder located on the medial and lateral portion of the wrist 3 to inflate the most, reducing slip between the patient and LID 400 due to the bladder holding on to critical anatomy features.

FIG. 5C illustrates an example bladder 440 that may line a top portion of a clamshell 430a, and thereby engage a posterior, medial and lateral sides of arm 2 and wrist 3 of a patient. Bladder 440 may be formed of a flexible material that contains selectively receives a fluid such as a liquid or gas. Bladder 440 may be formed of vinyl. Internal volume of bladder 440 may be interrupted by inflation limiting means 441. The inflation limiting means 441 may include a plurality of discrete sealed or adhered locations, having a boundary defined by non-sealed or non-adhered portions of the bladder. These discrete sealed locations may define a dot shape, including squares, circles or ovals for example and may define a quilting effect. These discrete sealing locations may define elongate lines that extend along the bladder 440, defining a bellowing effect. Elongate lines may be curved or straight. The discrete sealed inflation limiting means are preferable spaced away from the bladder outermost boundary, allowing flow of fluid around and into the entire bladder volume as define by this outermost sealed boundary of the bladder.

In FIG. 5C quilting dots, 242 are illustrated. Quilting dots 242 are preferably disposed along the portion of bladder 440 that engages a posterior portion of patient arm 2 without pinching on this portion of the arm, which may potentially cause numbness. Lateral and medial portions 445a, 445b may be free of inflation limiting means and therefore preferably inflate to further and engage lateral and medial portions of arm 2. This is similar to zone B in earlier disclosed embodiments. This may improve the resistance to slip due to rotational forces on the arm 2. In addition, end 443, which may be a flanged end of bladder 440 may also be free of inflation limiting means to improve grip around the wrist 3. Flanged end 443 may extend further around wrist 3 to improve grip. This increased inflation is similar to zone A as disclosed in earlier embodiments.

A drape (not shown) may be sealed into the clamshell 430 or handplate 420, which allows the fingers and hand to be unsterile when loading the limb into the device and the forearm can be draped using the drape attached to the clamshell. When closed, the clamshell may be held shut via integrated spring features that may be actuations to selectively open clamshell. In some embodiments, foam may be located within the bottom part of the clamshell 430b to engage the patient's skin. This foam may include a plurality of layers that may be selectively removed, one layer at a time. This may provide a level of adjustment to the foam height such that a layer of foam can be peeled off to accommodate a larger hand to fit properly inside the clamshell. In some embodiments, the handplate can be designed such that there are specific sides for the right and left limb with air bladders located on both sides.

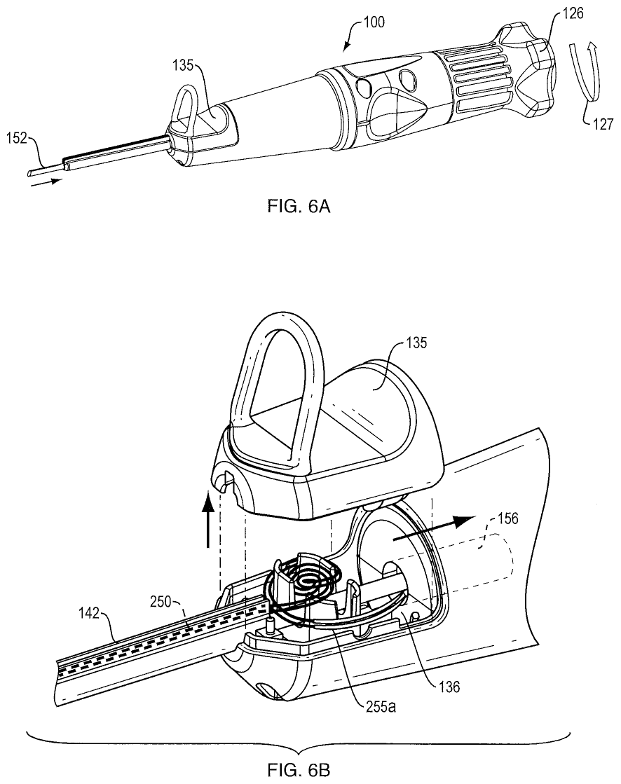

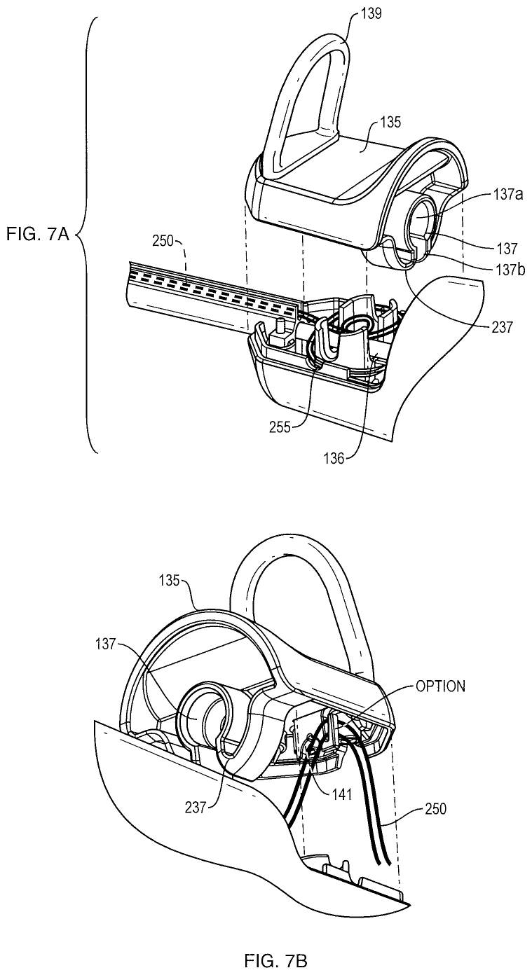

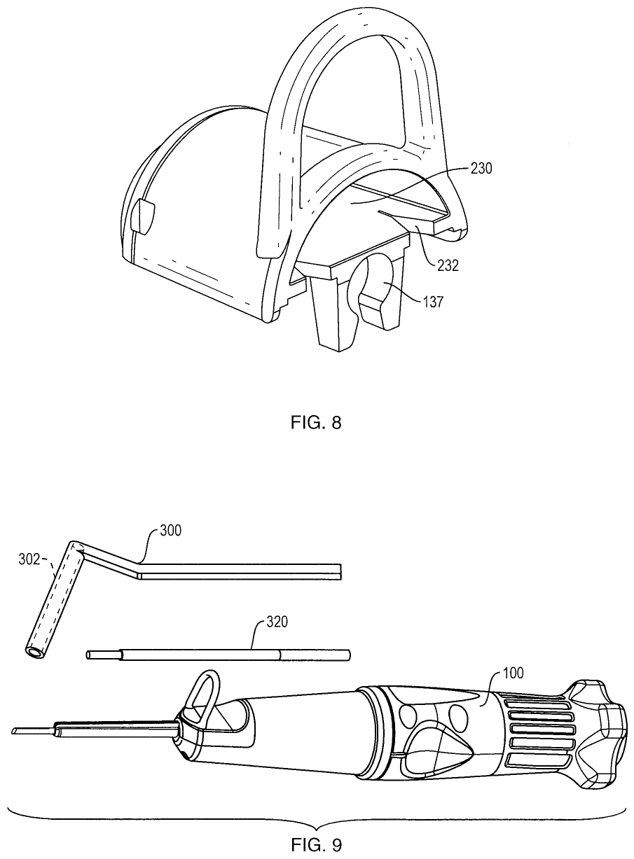

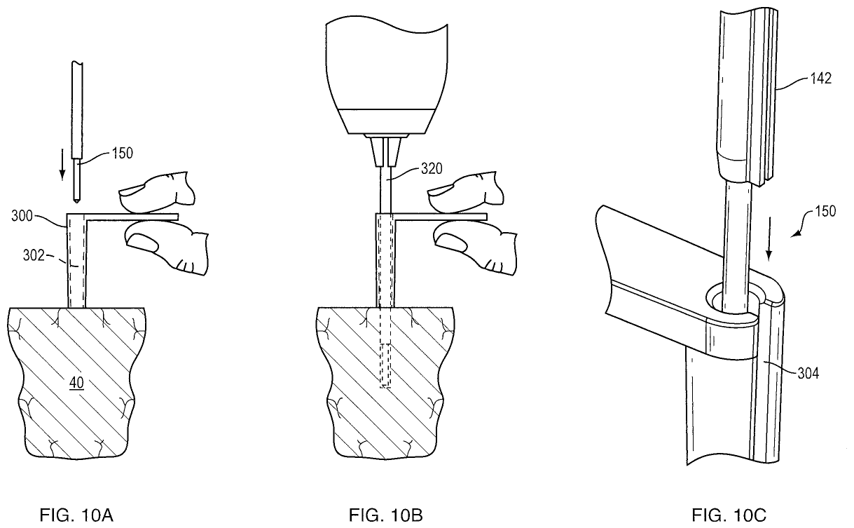

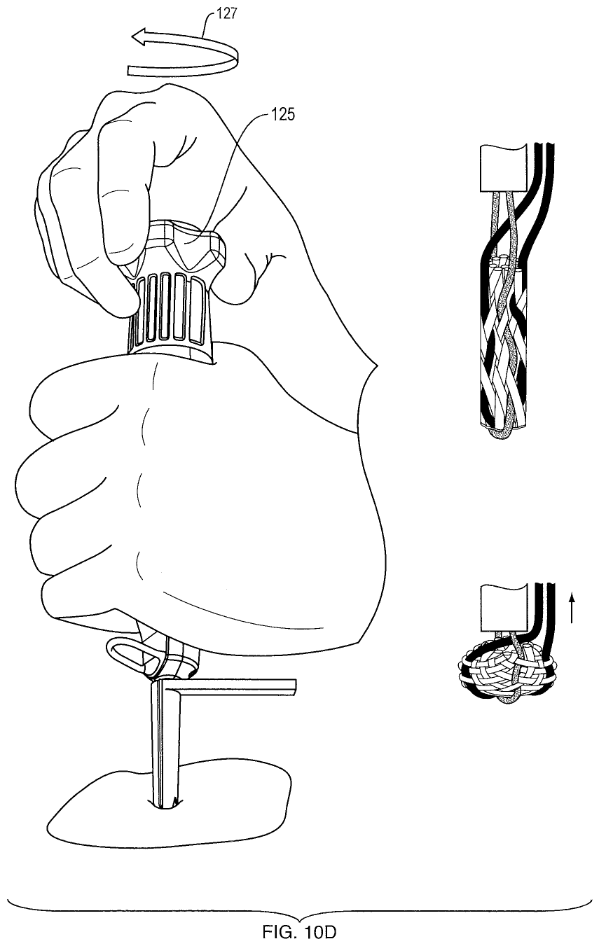

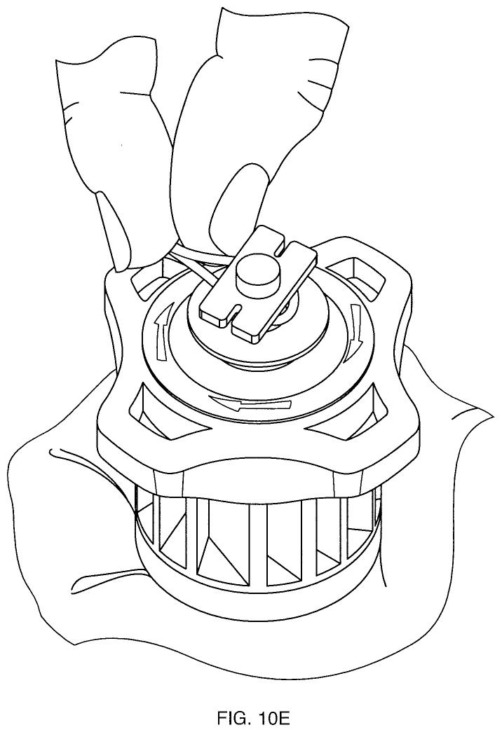

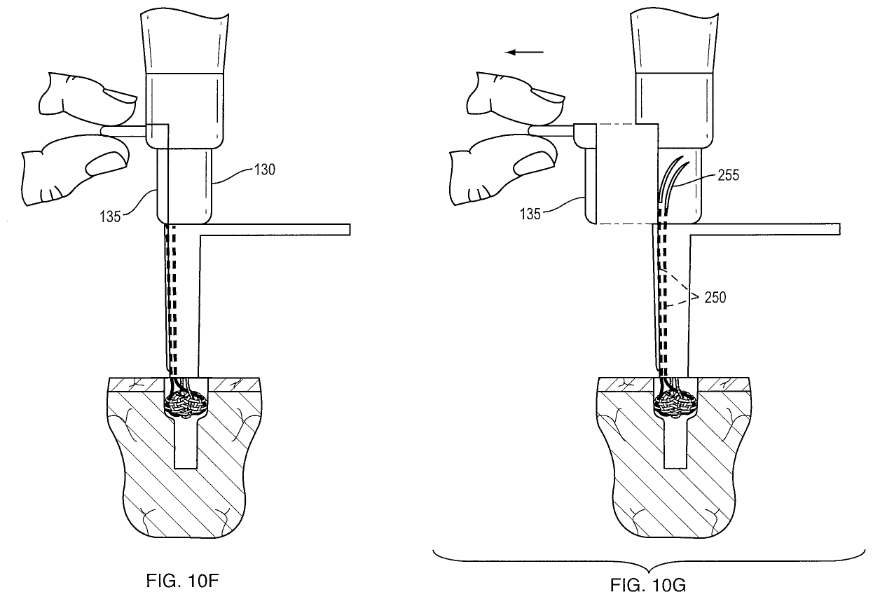

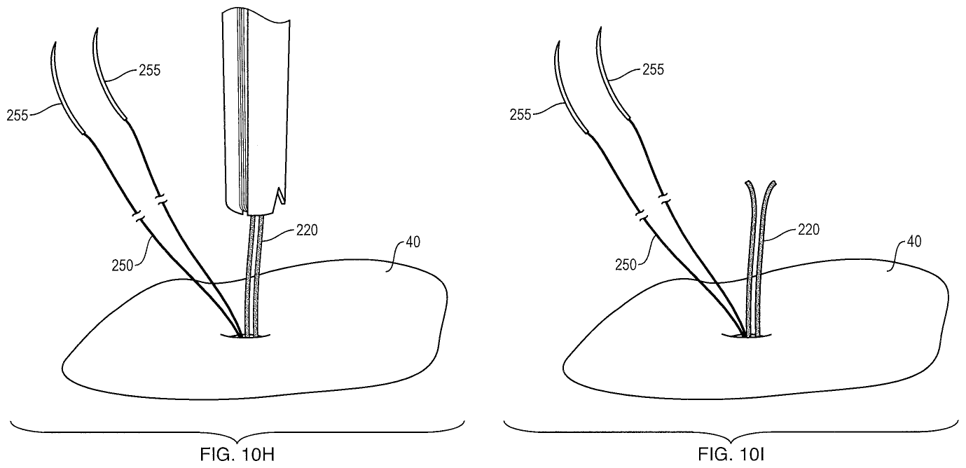

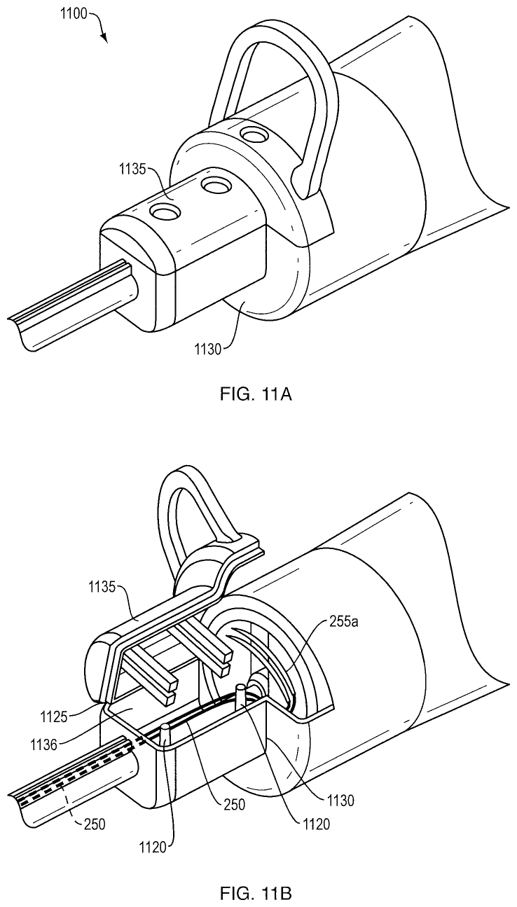

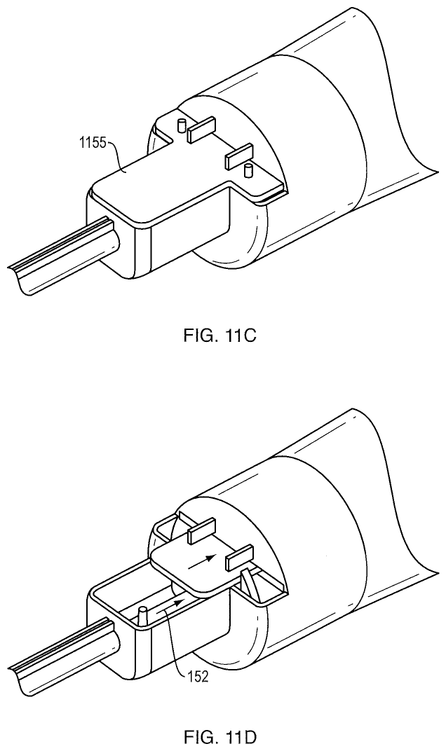

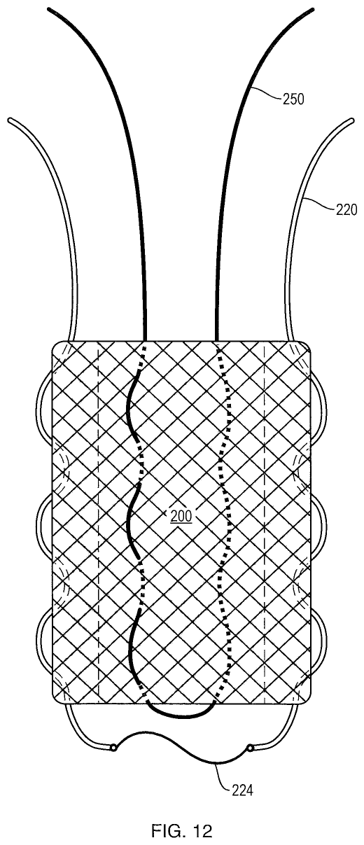

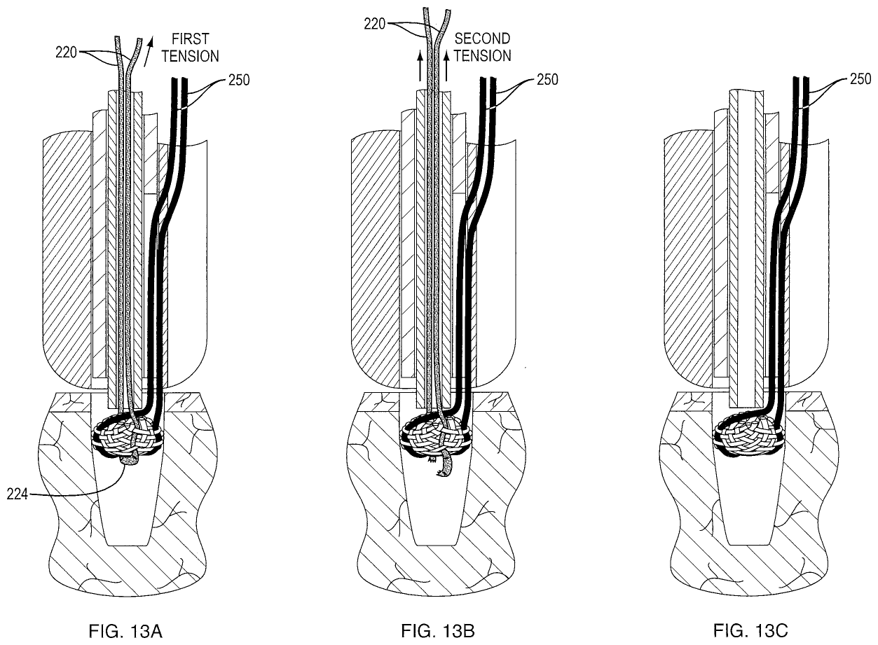

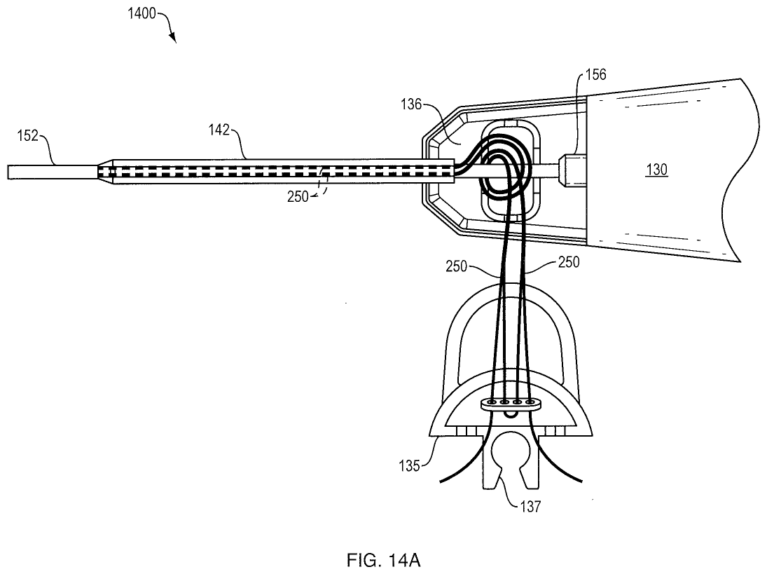

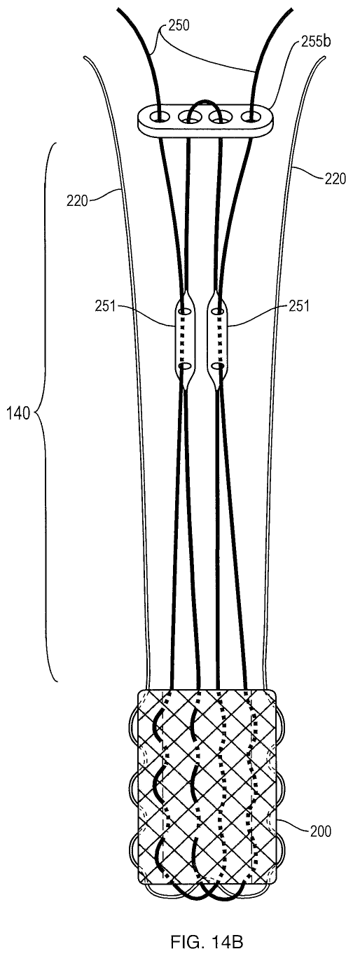

Illustrated in FIG. 6A-6B is another clamshell embodiment of a handplate 620 that may be lined at least partially with a bladder, such as bladder 440 (not shown). FIGS. 6A-6B illustrate the handplate 620 and clamshell only, with the bladder liner removed for simplicity of understanding. In this embodiment, connecting post 210 may be coaxial with an axial load (F) on the arm 2 in the lateral decubitus position. Clamshell top 630 may hinge open and lock with spring or snap mechanism 650. A force directed to open the clamshell, like the pressure created by inflating the bladder, creates a force to further drive the integrated mechanism 650 into the locked position. The integrated spring mechanism 650 may include two mechanisms, one each either side of the Clamshell 630. The clamshell top 620 will preferably not release when only one feature is released. Both mechanisms 650 are preferably released either individually or simultaneously to allow the clamshell to open. Both mechanisms may be released simultaneously by a single hand.

A method of stabilizing a patient limb may include placing a patient's hand on a LID handplate (220, 420) and encircling the patient arm 2 and wrist 3 with a sleeve, such as wrap 230, tube 330 or clamshell 430. In some example methods, the sleeve may be adjusted to tighten to the anatomy of the patient, reducing the amount of fluid that is required to inflate the bladder(s). In some example methods, the sleeve may be closed around the patient and may be secured via a first mechanical means such as a hook and loop (Velcro) or a temporary adhesive. When closed, the sleeve may be substantially conical in shape to match patient anatomy where the cross section of the patient's forearm is greater than the cross section of their wrist. In some example methods, a top portion of a clamshell may be hinged open to place the arm and wrist on the handplate, and then hinged closed and snapped closed around the arm and wrist. Arm and wrist may be adjusted to place the carpal area 7 adjacent a distal end of the sleeve. In some example methods, a sterile drape fixed to the LID may be drawn over the LID and patient arm. Pump 245 may actuated to inflate the bladder (240, 340, 440) once the arm 2 and wrist 3 are situated. Actuation of the pump may be manual. Bladder may include inflation limiting reducing the volume of fluid required to inflate the bladder. In the inflated configuration, the bladder may define a variable inflation profile, including a plurality of zones along the bladder surface that inflate to different degrees. Bladder may include inflation limiting means to constrain inflation along target zones of the bladder. When in the wrapped form that is generally conical or tubular, this variable inflation profile defines a variable internal opening size along the bladder 240, 340, 440. Inflating the bladder may for example minimally inflates a zone of the bladder adjacent the anterior surface of the wrist, to avoid hand numbness. Inflating the bladder 240, 340, 440 may inflate a zone of the bladder along the lateral and/or medial sides of the arm 2. A rotational force may then be applied to the patient arm for better access to the target joint, the increased inflation along the lateral and/or medial sides of the arm 2 configured to resist rotational slip of the LID relative to the patient arm. Inflating the bladder 240, 340, 440 may preferably inflate a zone of the bladder around the wrist 3 to a smaller internal opening size. An axial force may then be applied to the patient arm for better access to the target joint, which may distract the joint, the increased inflation around the wrist 3 configured to resist axial slip of the LID relative to the patient arm.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. An orthopedic limb interface device for positioning and retaining a lower portion of the arm and hand of the human body during orthopedic procedures, comprising:
a handplate; and
a sleeve coupled to the handplate, configured to surround and grippingly retain the arm, said sleeve including a bladder configured to inflate to an inflated configuration upon the introduction of a fluid therein and adjust an internal opening size of the sleeve; and wherein the bladder includes a plurality of inflatable zones including at least one zone of greater inflation and at least one zone of limited inflation along the bladder, the plurality of inflatable zones configured to improve retention of the orthopedic limb interface device with the arm.

2. The orthopedic limb interface device of claim 1 wherein the at least one zone of greater inflation is disposed along an end of the bladder, configured to engage around a wrist portion of the arm and improve a resistance to slip upon axial traction along a longitudinal axis of the arm.

3. The orthopedic limb interface device of claim 2 wherein the plurality of inflatable zones includes at least two discrete zones of greater inflation separated by at least one zone of limited inflation, the at least two discrete zones configured to engage opposing sides of an anterior surface of the arm when assembled around the arm, configured to improve a resistance to slip upon application of a rotational force to the arm.

4. The orthopedic limb interface device of claim 1 wherein the at least one zone of limited inflation is defined by means of limiting inflation including at least one of ribs, quilting or bellows.

5. The orthopedic limb interface device of claim 1 wherein the at least one zone of limited inflation is at least partially defined by a plurality of quilting dots.

6. The orthopedic limb interface device of claim 1 further comprising a sterile drape fixedly coupled thereto, configured to unfold and provide a sterile barrier over the arm, handplate and sleeve.

7. The orthopedic limb interface device of claim 1 wherein the bladder is operatively coupled to a fluid transport system, configured to transfer fluid into the bladder and thereby inflate the bladder, the fluid transport system including a pressure limiting valve.

8. An orthopedic limb interface device to position and retain a lower portion of the arm and hand of the human body during orthopedic procedures, comprising:
a handplate; and
a sleeve coupled to the handplate, configured to surround and grip the arm, said sleeve including a bladder configured to inflate upon the introduction of a fluid therein and adjust an internal opening size of the sleeve; and wherein the bladder includes a plurality of inflatable zones fluidly coupled to each other including at least one zone including inflation limiting means and at least one zone free of inflation limiting means configured to inflate further than the at least one zone including inflation limiting means, the plurality of inflatable zones configured to improve retention of the orthopedic limb interface device with the arm.

9. The orthopedic limb interface device of claim 8 wherein the at least one of the zones including inflation limiting means is configured to reduce pinching of the arm and thereby mitigate numbness of the hand.

10. The orthopedic limb interface device of claim 8 wherein the at least one zone free of inflation limiting means extends along a distal end of the bladder, configured to inflate and engage a wrist portion of the arm to improve a resistance to slip upon axial traction along a longitudinal axis of the arm.

11. The orthopedic limb interface device of claim 8 wherein the at least one zone free of inflation limiting means includes two discrete zones, each of the two discrete zones defining elongate zones extending axially along the bladder and separated by a zone including limited inflation means, the two discrete zones configured to improve a resistance to slip upon application of a rotational force to the arm.

12. The orthopedic limb interface device of claim 8 wherein the inflation limiting means includes at least one of ribs, quilting or bellows.

13. The orthopedic limb interface device of claim 8 wherein the inflation limiting means includes a plurality of quilting dots.

14. The orthopedic limb interface device of claim 8 further comprising a sterile drape operatively coupled thereto, configured provide a sterile barrier over the arm, handplate and sleeve.

15. The orthopedic limb interface device of claim 8 wherein the bladder is provided operatively coupled to a fluid transport system, configured to transfer fluid into the bladder and thereby inflate the bladder, the fluid transport system including a pressure limiting valve.

16. An orthopedic limb interface device to position and retain the lower portion of the arm and wrist of the human body during orthopedic procedures, comprising:
an inflatable bladder operatively coupled to a handplate, the inflatable bladder configured to encircle the arm and wrist and inflate upon the introduction of a fluid therein; and wherein the inflatable bladder includes zones with inflation limiting means and zones absent inflation limiting means so that the inflatable bladder defines a variable inflation profile, the variable inflation profile configured to increase retention of predetermined portions of the wrist and hand to rests slipping of the arm while under traction.

17. The orthopedic limb interface device of claim 16 wherein the variable inflation profile includes at least one zone absent inflation limiting means disposed along a distal edge of the inflatable bladder configured to engage around the wrist and retain the orthopedic limb interface device while undergoing axial traction.

18. The orthopedic limb interface device of claim 16 wherein the variable inflation profile includes at least two zones absent inflation limiting means, each of the at least two zones disposed along a longitudinal axis of the inflatable bladder configured to engage along a length of the arm and retain the orthopedic limb interface device for improved resistance to slip while undergoing rotational traction.

19. The orthopedic limb interface device of claim 16 wherein the inflation limiting means include a plurality of quilting dots.
